(19) United States
(12) Patent Application Publication
(10) Pub. No.: US 2014/0236050 A1
(43) Pub. Date: Aug. 21, 2014

(54) METHOD, APPARATUS, AND HIFU SYSTEM FOR GENERATING ULTRASOUND THAT FORM MULTI-FOCI IN REGION OF INTEREST

(71) Applicant: Samsung Electronics Co., Ltd., Suwon-si (KR)

(72) Inventors: Ho-taik LEE, Yongin-si (KR); Won-chul BANG, Seongnam-si (KR)

(21) Appl. No.: 14/025,049

(22) Filed: Sep. 12, 2013

(30) Foreign Application Priority Data
Feb. 15, 2013 (KR) 10-2013-0016602

(51) Int. Cl. A61N 7/02

(52) U.S. Cl. CPC A61N 7/02
USPC 601/3

(57) ABSTRACT

Provided is a method of generating ultrasound that forms focus points in a region of interest. Additional embodiments include related apparatuses and system embodiments. The method includes: setting focus patterns that each indicate focus points to be formed by radiating therapeutic ultrasound to a region of interest of an object; calculating a parameter of the therapeutic ultrasound based on characteristics of tissue in the region of interest existing on a path through which the therapeutic ultrasound moves; and generating therapeutic ultrasound set at different frequencies to form the focus patterns, based on the calculated parameter.

### CROSS-REFERENCE TO RELATED APPLICATIONS

This application claims the benefit of Korean Patent Application No. 10-2013-0016602 filed on Feb. 15, 2013, in the Korean Intellectual Property Office, the entire disclosure of which is incorporated herein by reference for all purposes.

### BACKGROUND

1. Field

The following description relates to methods, apparatuses, and high-intensity focused ultrasound (HIFU) systems for generating ultrasound energy that forms multiple focus points in a region of interest.

2. Description of the Related Art

As medical science has been developed, noninvasive surgery as well as minimally invasive surgery has recently been used to locally treat a tumor. Because invasive surgeries can be risky to a patient's health, alternative therapies that offer the ability to treat tumors are attractive because they provide ways to help treat tumors while avoiding the complications that can accompany more invasive surgeries. For example, surgeries can involve issues such as reactions to anesthesia or infections. Less invasive procedures avoid or minimize some of these issues, so there is considerable interest in noninvasive ways that treat tumors in effective ways.

Among noninvasive surgery methods, a High-intensity focused ultrasound (HIFU) treatment has been widely used since ultrasound is able to pass harmlessly through the human body, with the ultrasound being concentrated on an area that is to be destroyed by ablation. In the case of a HIFU treatment, the treatment is a method of necrotizing a lesion by focusing and radiating a high-intensity ultrasound to the lesion in the human body. Ultrasound focused and radiated to the lesion is converted into thermal energy at the region where the ultrasound is focused. The intensely concentrated thermal energy at the focused area causes coagulating necrosis of the lesion and blood vessels due to a temperature increase of a portion to which the ultrasound is radiated. Since the temperature is raised almost instantly, HIFU acts to effectively remove only the radiated portion while preventing heat from diffusing to surrounding areas of the radiated portion.

An HIFU apparatus includes a transducer (or a therapeutic ultrasound probe) that converts an electrical signal into ultrasound, and may control a position at which a focus is to be formed by adjusting a particle velocity in the transducer. A computer connected to the transducer or built into the transducer regulates the electric signal that the transducer converts into ultrasound so as to be able to change parameters of the ultrasound such as its intensity or frequency.

Some therapies use a method of simultaneously forming a plurality of focus points by using a transducer (or a therapeutic ultrasound probe), including a plurality of elements. However, current methods that use this approach lead to levels of side lobes or grating lobes that are so high that high heat may be generated in an undesired area, unlike a method of forming a single focus point.

### SUMMARY

Provided are methods, apparatuses, and high-intensity focused ultrasound (HIFU) systems for generating ultrasound that forms focus points in a region of interest.

Additional aspects will be set forth in part in the description which follows and, in part, will be apparent from the description, or may be learned by practice of the presented embodiments.

In one general aspect, a method of generating ultrasound includes setting focus patterns that each indicate focus points to be formed by radiating therapeutic ultrasound to a region of interest of an object, calculating a parameter of the therapeutic ultrasound based on characteristics of tissue in the region of interest existing on a path through which the therapeutic ultrasound moves, and generating therapeutic ultrasound set at different frequencies to form the focus patterns, based on the calculated parameter.

In the method, different frequencies may be set such that the intensity of a sound field at a point other than the region of interest after the focus patterns are formed is less than a predetermined critical value.

In the method, the different frequencies may be set such that at least one of positions or intensities of side lobes or grating lobes generated by the therapeutic ultrasound that forms the focus patterns are not the same.

In the method, the calculating of the parameter of the therapeutic ultrasound may include, calculating the parameter of the therapeutic ultrasound based on a density of the tissue, a speed of sound of the therapeutic ultrasound in the tissue, and a wave number of the therapeutic ultrasound in the tissue.

In the method, the parameter of the therapeutic ultrasound may be at least one of an amplitude and a phase of the therapeutic ultrasound.

The method may further include selecting the region of interest to which the therapeutic ultrasound is to be radiated, wherein the setting of the focus patterns comprises setting positions of focus points included in the focus patterns in the selected region of interest.

The method may further include determining an order in which the generated therapeutic ultrasound that forms the focus patterns is radiated.

In another general aspect, a non-transitory computer-readable recording medium has embodied thereon a program for executing the method just presented.

In another general aspect. an apparatus for generating ultrasound that forms focus points includes a focus pattern setter configured to set focus patterns each indicating focus points to be formed by radiating therapeutic ultrasound to a region of interest of an object, a parameter calculator configured to calculate a parameter of the therapeutic ultrasound based on characteristics of tissue in the region of interest existing on a path through which the therapeutic ultrasound moves, and an ultrasound generator configured to generate therapeutic ultrasound set at different frequencies to form the focus patterns, based on the calculated parameter.

In the apparatus, the ultrasound generator may be configured to set the different frequencies such that the intensity of a sound field observed at a point other than the region of interest after the focus patterns are formed is less than a critical value.

In the apparatus, the ultrasound generator may be configured to set the different frequencies such that at least one of positions or intensities of side lobes or grating lobes generated by the plurality of therapeutic ultrasounds that forms the focus patterns are not the same.

In the apparatus, the parameter calculator may be configured to calculate the parameter of the therapeutic ultrasound based on a density of the tissue, a speed of sound of the therapeutic ultrasound in the tissue, and a wave number of the therapeutic ultrasound in the tissue.

In the apparatus, the parameter of the therapeutic ultrasound may be at least one of an amplitude and a phase of the therapeutic ultrasound.

The apparatus may further include a region of interest selector configured to select the region of interest to which the therapeutic ultrasound is to be radiated, wherein the focus pattern setter is configured to set positions of different focus points included in the focus patterns within the selected region of interest.

The apparatus may further include an order determiner that is configured to determine an order in which the generated therapeutic ultrasound that forms the focus patterns is radiated.

### BRIEF DESCRIPTION OF THE DRAWINGS

These and/or other aspects will become apparent and more readily appreciated from the following description of the embodiments, taken in conjunction with the accompanying drawings.

FIG. 1 is a diagram illustrating a high-intensity focused ultrasound (HIFU) system according to an embodiment.

FIG. 2 is a diagram illustrating a therapeutic ultrasound probe according to an embodiment.

FIGS. 3A through 3F illustrate focus patterns according to an embodiment.

FIG. 4 is a diagram illustrating a relationship between a focus and a therapeutic ultrasound probe, according to an embodiment.

FIG. 5 is a diagram for explaining a process performed by a parameter calculator to calculate a sound pressure of therapeutic ultrasound by using an angular spectrum method (ASM), according to an embodiment.

FIGS. 6A through 6C are graphs illustrating therapeutic ultrasounds according to an embodiment.

FIG. 7 is a diagram illustrating a central workstation according to another embodiment.

FIG. 8 is a diagram illustrating the HIFU system according to another embodiment.

FIG. 9 is a flowchart illustrating a method performed by the central workstation to generate ultrasound that forms focus points in a region of interest, according to an embodiment.

### DETAILED DESCRIPTION

The following detailed description is provided to assist the reader in gaining a comprehensive understanding of the methods, apparatuses, and/or systems described herein. However, various changes, modifications, and equivalents of the systems, apparatuses and/or methods described herein will be apparent to one of ordinary skill in the art. Also, descriptions of functions and constructions that are well known to one of ordinary skill in the art may be omitted for increased clarity and conciseness.

Throughout the drawings and the detailed description, the same reference numerals refer to the same elements. The drawings may not be to scale, and the relative size, proportions, and depiction of elements in the drawings may be exaggerated for clarity, illustration, and convenience.

The features described herein may be embodied in different forms, and are not to be construed as being limited to the examples described herein. Rather, the examples described herein have been provided so that this disclosure will be thorough and complete, and will convey the full scope of the disclosure to one of ordinary skill in the art.

The present invention now will be described more fully hereinafter with reference to the accompanying drawings, in which elements of the invention are shown. The present invention may, however, be embodied in many different forms and should not be construed as limited to the exemplary embodiments set forth herein. Rather, these embodiments are provided so that this disclosure will be thorough and complete, and will fully convey the scope of the present invention to one of ordinary skill in the art. Numerous modifications and adaptations will be readily apparent to one of ordinary skill in this art from the detailed description and the embodiments without departing from the spirit and scope of the present invention.

As used herein, the term “and/or” includes any and all combinations of one or more of the associated listed items. Expressions such as “at least one of,” when preceding a list of elements, modify the entire list of elements and do not modify the individual elements of the list.

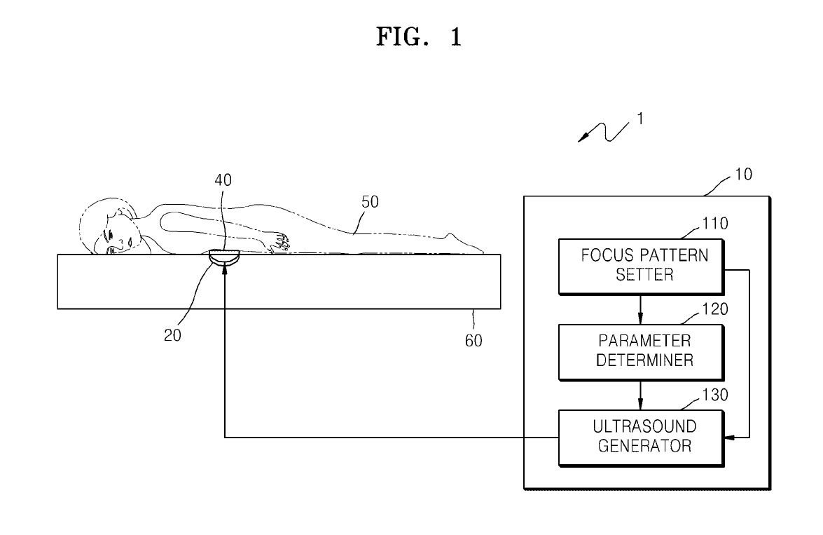

FIG. 1 is a diagram illustrating a high-intensity focused ultrasound (HIFU) system 1 according to an embodiment.

Referring to FIG. 1, the HIFU system 1 includes a central workstation 10 and a therapeutic ultrasound probe 20. In this example, the central workstation 10 includes a focus pattern setter 110, a parameter calculator 120, and an ultrasound generator 130. As shown in FIG. 1, the central workstation 10 is communicatively coupled to the therapeutic ultrasound probe 20. While FIG. 1 has portrayed the central workstation 10 and the therapeutic ultrasound probe 20 as being separate from one another, in other embodiments their functionality uses an integrated architecture.

The central workstation 10 transmits an electrical signal to the therapeutic ultrasound probe 20, allowing it to set various parameters of the ultrasound signal by modifying characteristics of the electric signal sent to drive the therapeutic ultrasound probe 20, thereby changing the characteristics of the ultrasound emitted by the therapeutic ultrasound probe 20.

Only elements of the central workstation 10 that are related to the present embodiment are shown in FIG. 1. Accordingly, it would be understood by one of ordinary skill in the art that the central workstation 10 may further include general-purpose elements other than the elements shown in FIG. 1, in other embodiments.

Also, each of the focus pattern setter 110, the parameter calculator 120, and the ultrasound generator 130 of the central workstation 10 of FIG. 1 includes and uses one or more processors. A processor may include an array of logic gates, or a combination of a general-purpose microprocessor and a program that may be executed by the microprocessor. Alternatively, it would be understood by one of ordinary skill in the art that the processor includes any of other types of hardware that process data. For example, the processor might include a custom processor, a multicore processor, or a parallel or distributed processor.

The therapeutic ultrasound probe 20 radiates therapeutic ultrasound to a region of interest in an object 50. Part of the operation of the therapeutic ultrasound probe 20 is that it needs to operate so that it can focus the ultrasound to ablate the desired portion of the object 50 for treatment purposes. In an example, the therapeutic ultrasound refers to high-intensity focused ultrasound (HIFU), but it is not limited thereto. For example, other types of ultrasound or related radiation may be used if they can provide similar treatment functionality.

In a use case for one embodiment, the therapeutic ultrasound probe 20 is provided in a bed 60 on which the object 50 lies down, and may remove a lesion in the region of interest by radiating therapeutic ultrasound to the region of interest in the body of the object 50. In this case, a gel pad 40 that may help to transmit therapeutic ultrasound may be disposed between the object 50 and the bed 60. For example, as discussed above, the therapeutic ultrasound probe 20 focuses a short, intense burst of ultrasound, optionally thorough a gel pad 40, into the body of the object 50. The ultrasound burst heats a tumor and kills the tumor, but does not harm other tissues in the region surrounding the tumor.

Although the therapeutic ultrasound probe 20 is provided in the bed 60 in FIG. 1, the present embodiment is not limited thereto. In one example, the therapeutic ultrasound probe 20 is provided above the object 50, and radiates therapeutic ultrasound downwards to the object 50. Alternatively, the therapeutic ultrasound probe 20 is positioned to one side of the object 50. The therapeutic ultrasound probe 20 emits ultrasound as discussed above to achieve a clinical outcome.

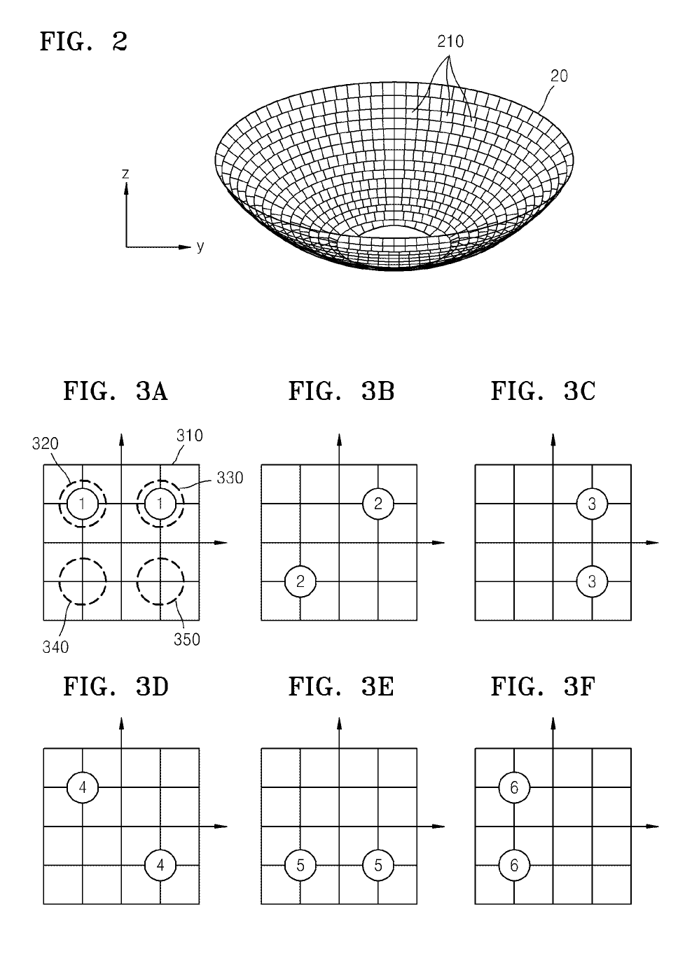

FIG. 2 is a diagram illustrating the therapeutic ultrasound probe 20 according to an embodiment.

Referring to FIG. 2, the therapeutic ultrasound probe 20 may be formed such that one or more elements 210 are arranged on a disc-shaped support plate with a recessed center. When the therapeutic ultrasound probe 20 includes a plurality of elements 210, the elements 210 may be set to receive a signal transmitted from the central workstation 10 (see FIG. 1), individually radiate therapeutic ultrasounds, and radiate therapeutic ultrasounds at different times. Because the orientation and positioning, as well as timing, of each of the elements 210 differ slightly from one another, each of the radiated therapeutics signals from the elements 210 emits ultrasound energy to a slightly different portion of the object 50, allowing the therapeutic ultrasound probe 20 to control where the ultrasound energy is directed as well as characteristics of the energy.

That is, since the elements 210 individually radiate ultrasounds, in an embodiment when a position of the therapeutic ultrasound probe 20 is fixed, focus points at which the ultrasounds are focused are different and multiple focus points may be formed.

In detail, each of the elements 210 may convert an electrical signal having a predetermined amplitude and a predetermined phase input from the central workstation 10 (see FIG. 1) into an ultrasound signal having a predetermined intensity and a predetermined phase, and may output the ultrasound signal. For example, the therapeutic ultrasound probe 20 may define a known relationship between the predetermined amplitude and phase input from the electrical signal to determine these values for the ultrasound. Each of the elements 210 are manufactured as, for example, a piezoelectric transducer. A piezoelectric transducer changes an electrical signal to a mechanical signal. If a piezoelectric transducer is used, the electrical signal causes the transducer to vibrate, and the mechanical vibrations emit sound, which is set at a frequency corresponding with ultrasound, which varies depending on what type of HIFU therapy is desired.

Ultrasound generated by each of the elements 210 may be focused on the region of interest in the body of the object 50 (see FIG. 1). The ultrasound focused on the region of interest may be converted into thermal energy to increase a temperature of the region of interest and necrotize a lesion in the region of interest. For example, increasing the temperature of the region kills the tissue in that region without collateral damage. The region of interest may correspond to tissue such as a breast, a liver, or the abdomen including a lesion, but it is not limited thereto. The therapies described herein apply to cancerous tissue, but it may be used to ablate other tissue if it is so desired.

Also, the therapeutic ultrasound probe 20 of FIG. 2 is just an embodiment of the present invention, and it would be understood by one of ordinary skill in the art that various modifications of the therapeutic ultrasound probe 20 are within the scope of the present invention. For example, certain differences such as a different size or shape of the therapeutic ultrasound probe 20 or different characteristics of the elements 210 may be present while remaining valid embodiments.

Referring back to FIG. 1, the central workstation 10 sets focus patterns each indicating a group of focus points to be formed by radiating therapeutic ultrasound to the region of interest. By using a group of focus points for the ultrasound, it is possible to treat multiple smaller areas so as to reduce treatment time while also targeting tissue more accurately to avoid problems that can result from border areas and overlap. To address some of these issues, central workstation 10 generates therapeutic ultrasounds having different frequencies that form the focus patterns based on a parameter of the therapeutic ultrasound calculated by using characteristics of tissue constituting the region of interest. By using therapeutic ultrasound with different frequencies, it becomes easier to avoid interaction between multiple ultrasound signals. Functions of the focus pattern setter 110, the parameter calculator 120, and the ultrasound generator 130 included in the central workstation 10 will now be explained in detail.

The focus pattern setter 110 sets focus patterns each indicating a group of focus points to be formed by radiating therapeutic ultrasound to the region of interest. The focus points refer to a plurality of focus points formed by focusing therapeutic ultrasound on the region of interest. That is, unlike a single focus point that refers to one focus point formed when the therapeutic ultrasound probe 20 radiates therapeutic ultrasound one time, focus points refer to focus points simultaneously formed when the therapeutic ultrasound probe 20 radiates therapeutic ultrasound one time. Also, each focus pattern refers to a group of focus points that are included in the focus points and have different positions. Thus, focus pattern setter 110 is able to manage how the ultrasound is targeted and dispersed. Additionally, the focus pattern setter 110 provides the ability to speed up the ultrasound process by targeting groups of therapy targets with each pulse.

FIGS. 3A through 3F illustrate focus patterns according to an embodiment. Referring to FIGS. 3A through 3F, although a region of interest 310 of the object 50 (see FIG. 1) has a latticed square shape for convenience of explanation, the present embodiment is not limited thereto, and other shapes are usable with appropriately different configurations. Also, although the number of focus points included in each focus pattern is 2, the present embodiment is not limited thereto, and in some embodiments even more focus points provide even greater ability to speed up and control ultrasound therapy.

The focus pattern setter 110 (see FIG. 1) sets focus patterns each indicating a group of focus points to be formed in the region of interest 310. For example, the focus pattern setter 110 (see FIG. 1) may determine that 4 focus points 320 through 350 having different positions may be able to be formed in the region of interest 310, and may select two focus points from among the 4 focus points 320 through 350 and may set focus patterns (see FIGS. 3A through 3F) each indicating a group of the 2 selected focus points. The number of foci determined by the focus pattern setter 110 (see FIG. 1) is not limited to 4. Other embodiments may use more or fewer foci than 4, and determine how to locate the foci appropriately.

In detail, the focus pattern setter 110 (see FIG. 1) may calculate the number of focus patterns set by selecting 2 focus points from among 4 focus points as ₄C₂=6, and may set 6 patterns (see FIGS. 3A through 3F) in total. The determined 6 patterns (see FIGS. 3A through 3F) show different combinations of the focus points 320 through 350. Thus, the choice of foci and their locations is resolved through appropriate combinatoric analysis.

Referring back to FIG. 1, the focus pattern setter 110 transmits information about the set focus patterns to the parameter calculator 120 and the ultrasound generator 130. Although not shown in FIG. 1, the central workstation 10 may include a separate storage (not shown), and the focus pattern setter 110 may transmit the information about the set focus patterns to the storage, and the storage may store the information therein for future retrieval and usage. Storing the information may reduce future resource burden by avoiding the need to recalculate focus patterns every time they are desired for use.

The parameter calculator 120 calculates a parameter of therapeutic ultrasound by using characteristics of tissue in the region of interest existing on a path through which the therapeutic ultrasound moves. The path through which the therapeutic ultrasound moves refers to a path through which the therapeutic ultrasound radiated from the therapeutic ultrasound probe 20 moves to focus points. The path for the ultrasound is considered by the parameter calculator 120, because the ultrasound may propagate in different ways depending on the path and hence considering the path facilitates targeting the ultrasound. The focus points may be obtained by sharing the information about the set focus patterns transmitted from the focus pattern setter 110 to the parameter calculator 120.

In detail, the parameter calculator 120, in an embodiment, calculates a parameter of the therapeutic ultrasound by combining a density of tissue existing on a path through which the therapeutic ultrasound moves, a speed of sound of the therapeutic ultrasound in the tissue, and a wave number of the therapeutic ultrasound in the tissue. The parameter of the therapeutic ultrasound may refer to at least one of an amplitude and a phase of the therapeutic ultrasound. This information can be used by the parameter calculator to 120 to help provide the appropriate amount of therapeutic ultrasound to the proper destination.

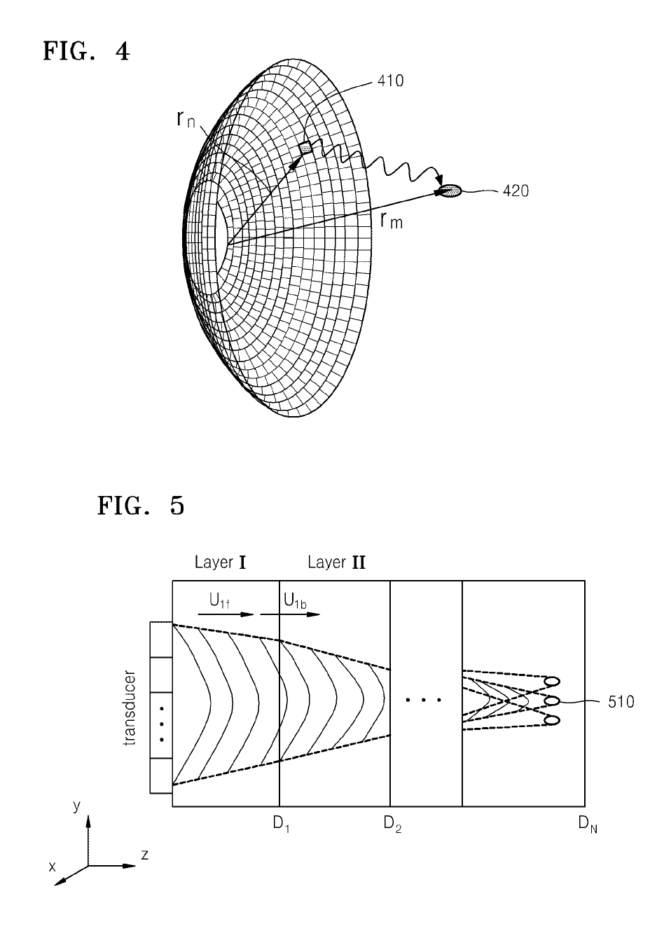

FIG. 4 is a diagram illustrating a relationship between the therapeutic ultrasound probe 20 (see FIG. 1) and a focus, according to an embodiment.

It is assumed that N elements exist in the therapeutic ultrasound probe 20 (see FIG. 1) and therapeutic ultrasound radiated by the therapeutic ultrasound probe 20 (see FIG. 1) forms focus points at M target positions. FIG. 4 illustrates a position vector of each focus point and one element included in the therapeutic ultrasound probe 20 (see FIG. 1) including the N elements. Referring to FIG. 4, rₙ is a position vector of an nth (n=1, 2, . . . , N) element 410, and rₘ is a position vector of an mth (m=1, 2, . . . , M) focus point 420.

In this case, the parameter calculator 120 (see FIG. 1) may calculate a sound pressure “p” applied by the N elements to the mth focus point 420 as a result of the therapeutic ultrasound by using a Rayleigh-Sommerfeld integral as shown in Equation 1. However, a method performed by the parameter calculator 120 (see FIG. 1) to calculate the sound pressure “p” is not limited to using the Rayleigh-Sommerfeld integral. In some embodiments, other similar methods are used, or any other method that provides a measure of p(rₘ).

$$p ( {r}_{m} ) = \frac{j ρ ck}{2 π} {\sum }_{n = 1}^{N} {u}_{n} {\int }_{Sn}^{ } \frac{{e}^{- j k \vert {r}_{m} - {r}_{n} \vert}}{\vert {r}_{m} - {r}_{n} \vert} d {S}_{n} \qquad Equation 1$$

In Equation 1, p, c , and k are a density of tissue, a speed of sound of therapeutic ultrasound in the tissue, and a wave number, respectively. Sₙ is a cross-sectional area of the nth element 410. Also, uₙ is a particle velocity in the nth element 410, and p(rₘ) is a sound pressure at a focus point having the position vector rₘ. These various values provide sufficient information to measure p(rₘ) using the Rayleigh-Sommerfeld integral.

A Rayleigh-Sommerfeld integral is most useful when media existing on a path through which therapeutic ultrasound moves are homogeneous. That is, when a plurality of tissues are distributed on a path through which therapeutic ultrasound moves and thus media are heterogeneous, the Rayleigh-Sommerfeld integral may fail to provide an accurate sound pressure. In this case, other methods may need to be used.

Accordingly, when media on a path through which therapeutic ultrasound moves are heterogeneous, the parameter calculator 120 (see FIG. 1) may calculate the sound pressure p(rₘ) at the focus point having the position vector rₘ by using an angular spectrum method (ASM). The ASM is a method of expanding a complex wave field to a summation of infinite number of plane waves.

FIG. 5 is a diagram for explaining a process performed by the parameter calculator 120 (see FIG. 1) to calculate a sound pressure of therapeutic ultrasound by using an ASM, according to an embodiment. The diagram shows how the ASM technique is analyze transmission of the ultrasound through multiple materials.

Referring to FIG. 5, when a plurality of different media (for example, internal body tissues) exist on a path through which therapeutic ultrasound moves, the parameter calculator 120 (see FIG. 1) calculates a transmitted sound field u_{1b} that passes through a boundary surface (that is, a discontinuous boundary surface) D₁ of the media by combining a transmission coefficient T with an incident sound field u_{1f} that is incident on the boundary surface. Next, the parameter calculator 120 (see FIG. 1) calculates U_{1b} that is an angular spectrum on a plane D₁ by performing a two-dimensional (2D) Fourier transform on the transmitted sound field uₘ. Next, the parameter calculator 120 (see FIG. 1) calculates U2 by correcting a phase change due to a distance difference between the plane D₁ and a plane D₂ based on the angular spectrum U_{1b}. Next, the parameter calculator 120 (see FIG. 1) calculates a sound field u2 on the plane D2 by performing a 2D Fourier inversion transform on the U2. By performing such calculation and analysis, the parameter calculator 120 corrects for different ultrasound transmission characteristics of different media.

The parameter calculator 120 (see FIG. 1) calculates the sound pressure p(rₘ) at a focus point 510 having the position vector rₘ) by repeatedly performing the above steps by as many as the number of boundary surfaces existing on a path through which therapeutic ultrasound moves. Such steps assess the overall effects of different ultrasound transmission media and uses them to target the ultrasound.

However, when media existing on a path through which therapeutic ultrasound moves are heterogeneous, a method performed by the parameter calculator 120 (see FIG. 1) to calculate the sound pressure “p” is not limited to an ASM. Other embodiments use other appropriate methods.

Referring back to FIG. 4, the parameter calculator (see FIG. 1) calculates a relationship between a particle velocity in an nth element and a sound pressure applied to an mth target position, that is, propagation characteristics of therapeutic ultrasound, by using Equation 2. The particle velocity refers to an amplitude and a phase of therapeutic ultrasound radiated by each element.

$$H ( m , n ) = \frac{jρ ck}{2 π} {\int }_{Sn}^{ } \frac{{e}^{- j k \vert {r}_{m} - {r}_{n} \vert}}{\vert {r}_{m} - {r}_{n} \vert} d {S}_{n} \qquad Equation 2$$

From Equations 1 and 2, Equation 3 that is a relationship between a matrix u of particle velocities of n elements, a matrix p of sound pressures applied to m target positions, and a matrix H of propagation characteristics of therapeutic ultrasounds may be obtained.

$$[ \begin{matrix}{H}_{( 1 , 1 )} & {H}_{( 1 , 2 )} & {H}_{( 1 , 3 )} & … & {H}_{( 1 , n )} \\ {H}_{( 2 , 1 )} & {H}_{( 2 , 2 )} & {H}_{( 2 , 3 )} & … & {H}_{( 2 , n )} \\ ⋮ & ⋮ & ⋮ & ⋮ & ⋮ \\ {H}_{( m , 1 )} & {H}_{( m , 2 )} & {H}_{( m , 3 )} & … & {H}_{( M , N )}\end{matrix} ] [ \begin{matrix}{u}_{1} \\ {u}_{2} \\ ⋮ \\ {u}_{N}\end{matrix} ] = [ \begin{matrix}{p}_{1} \\ {p}_{2} \\ ⋮ \\ {p}_{M}\end{matrix} ] \qquad Equation 3$$

The parameter calculator 120 (see FIG. 1) may obtain a particle velocity in each element needed to apply a desired sound pressure by using a pseudoiinverse method according to Equations 4 through 6. The particle velocity refers to an amplitude and a phase of ultrasound radiated by each element constituting the therapeutic ultrasound probe 20 (see FIG. 1).

Hu=p Equation 4

u=H⁺p Equation 5

u=H*^{t}(HH*^{t})⁻¹p Equation 6

In Equation 5, H⁺ is a pseudoiinverse matrix, and in Equation 6, H*^{t} is a conjugate transpose matrix.

Referring back to FIG. 1, the parameter calculator 120 transmits information about the calculated parameter to the ultrasound generator 130. Also, although not shown in FIG. 1, the central workstation 10 may include a separate storage (not shown), and the parameter calculator 120 may transmit the information about the calculated parameter to the storage, and the storage may store the information therein. By so doing, it allows the use of pregenerated results to avoid the need for processing when performing its functionality.

The ultrasound generator 130 forms focus patterns and therapeutic ultrasounds having different frequencies based on the calculated parameter, received from the parameter calculator 120. In some embodiments, frequencies are set such that a sound field observed at a point other than the region of interest after the focus patterns are formed is less than a predetermined critical value.

In an example, the frequencies are be set such that positions or intensities of side lobes or grating lobes generated by the therapeutic ultrasounds that form the focus patterns are not the same. By coordinating frequencies, multiple therapeutic ultrasound pulses are able to treat larger areas more quickly while minimizing side effects.

The frequencies may be automatically set by the ultrasound generator 130 without a user's intervention, or may be set by using information about frequencies input from the outside through an interface (not shown) provided on the central workstation 10. Additionally, the frequencies may be set to establish parameters that have trade-offs, such as establishing a balance of speed and minimizing side-effects.

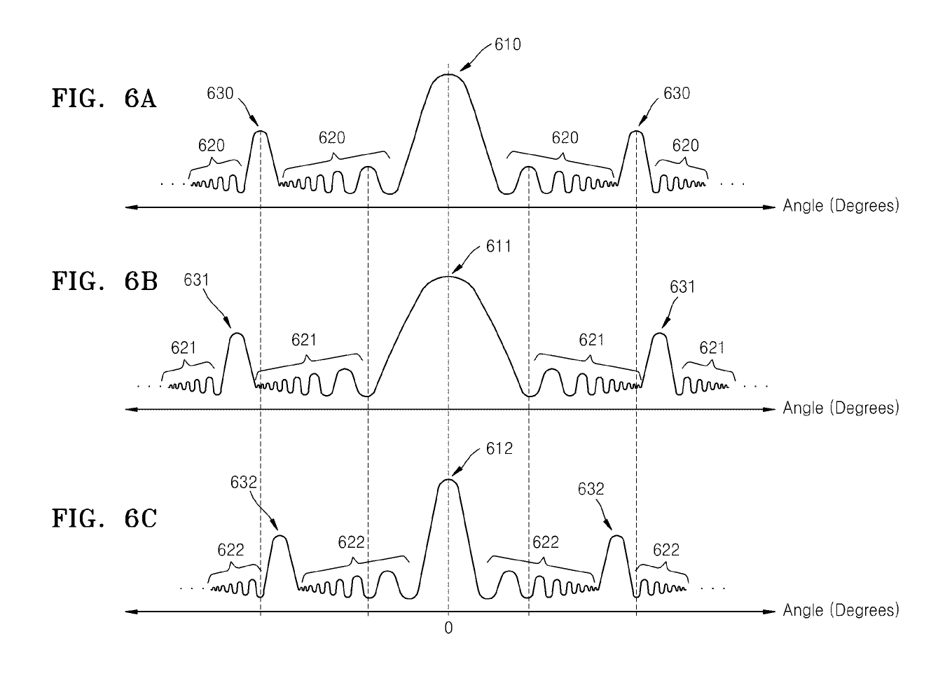

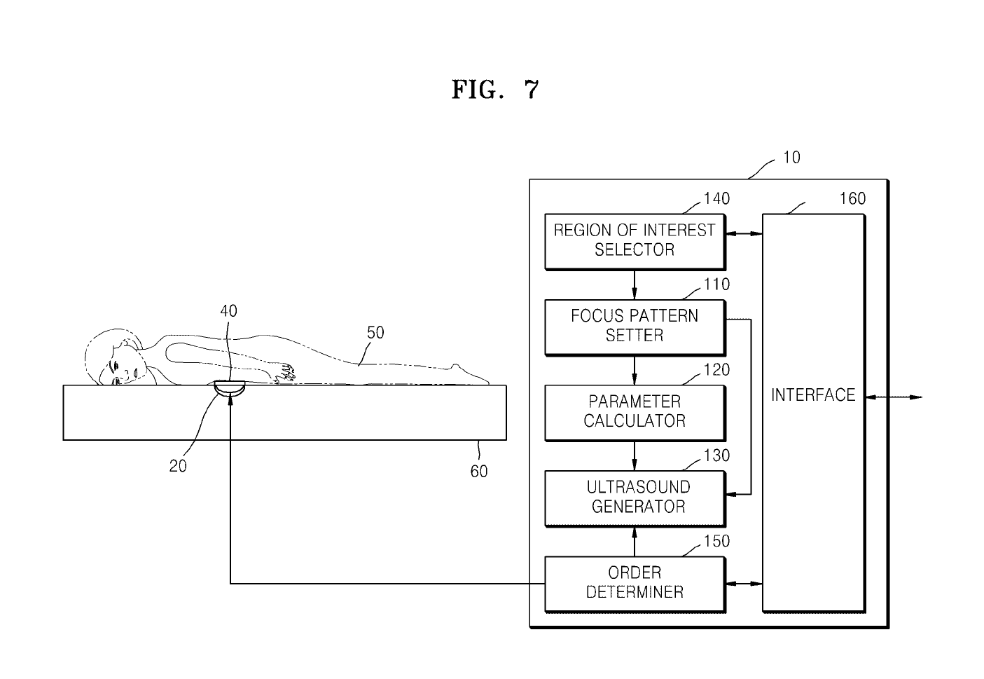

FIGS. 6A through 6C are graphs illustrating therapeutic ultrasounds having different frequencies, according to an embodiment

Referring to FIGS. 6A through 6C, the therapeutic ultrasounds have the same amplitude and the same phase and different frequencies. In FIGS. 6A through 6C, reference numerals 610, 611, and 612 denote main lobes of the therapeutic ultrasounds, and 620, 621, and 622 denote side lobes of the therapeutic ultrasounds. Also, reference numerals 630, 631, and 632 denote grating lobes of the therapeutic ultrasounds.

A side lobe or a grating lobe may amplify a therapeutic ultrasound signal at a position other than a focus point. In detail, the therapeutic ultrasound probe 20 (see FIG. 1) has a structure in which a plurality of elements are periodically repeated at predetermined intervals. When an interval between elements which are periodically repeated is greater than λ/2 (λ is a wavelength of therapeutic ultrasound), therapeutic signals generated by the elements may interfere with one another, and a phenomenon in which a therapeutic ultrasound signal is amplified at a position other than a focus point due to the interference may occur. This amplification may reduce treatment safety because it may cause ultrasound to heat areas which are not desired targets of the therapeutic ultrasound.

A HIFU apparatus generates heat by forming a focus of a therapeutic ultrasound signal at a position of a lesion, and treats the lesion by using the heat. With sufficient focused ultrasound, the wave energy from the ultrasound is transformed into thermal energy. However, when the phenomenon occurs, a therapeutic ultrasound signal may be amplified at a point of another organ other than a position of a lesion. In this case, tissue of the other organ is damaged due to heat generated by the therapeutic ultrasound signal, thereby reducing the safety of a HIFU treatment.

Accordingly, the ultrasound generator 130 (see FIG. 1) generates therapeutic ultrasound signals having the same amplitude and the same phase and different frequencies so that side lobes or grating lobes of the therapeutic ultrasound signals do not overlap with one another. For example, when it is assumed that the parameter calculator 120 (see FIG. 1) calculates a parameter of therapeutic ultrasound of FIG. 6A, the ultrasound generator 130 (see FIG. 1) generates therapeutic ultrasound signals having the same amplitude and the same phase and different frequencies as and from those of the therapeutic ultrasound of FIG. 6A, as shown in FIGS. 6B and 6C.

Referring to FIGS. 6A through 6C, main lobes of three ultrasound signals overlap but side lobes or grating lobes of the three ultrasound signals do not overlap. Accordingly, even when the therapeutic ultrasound probe 20 (see FIG. 1) radiates the three ultrasound signals simultaneously or at short intervals, a phenomenon in which a therapeutic ultrasound signal is amplified at a position other than positions of focus points may be avoided.

Referring back to FIG. 1, the ultrasound generator 130 transmits information about the therapeutic ultrasounds and their different frequencies and information about the focus patterns to the therapeutic ultrasound probe 20. The therapeutic ultrasound probe 20 radiates therapeutic ultrasound so that focus points included in the focus patterns are formed in the region of interest of the object 50 by using the information transmitted from the ultrasound generator 130. Thus, the therapeutic ultrasound will be emitted so that the ultrasound focuses only at the focus points.

Also, the ultrasound generator 130 may generate information about with how much time difference the focus patterns are repeatedly formed in the region of interest. By changing timing, it is possible to take account the role of timing on which areas will receive the most exposure to the therapeutic ultrasound. For example, the ultrasound generator 130 may generate information about with how much time difference the focus patterns shown in FIGS. 3A through 3F are repeatedly formed in the region of interest. With this information, it is possible to direct the ultrasound with greater control. In embodiments, the generated information is transmitted to the therapeutic ultrasound probe 20, and the therapeutic ultrasound probe 20 radiates therapeutic ultrasound that form focus points included in each of the focus patterns by using the transmitted information.

FIG. 7 is a diagram illustrating the central workstation 10 according to another embodiment.

Referring to FIG. 7, the central workstation 10 includes the focus pattern setter 110, the parameter calculator 120, the ultrasound generator 130, a region of interest selector 140, an order determiner 150, and an interface 160. Only elements of the central workstation 10 that are related to the present embodiment are shown in FIG. 7. Accordingly, it would be understood by one of ordinary skill in the art that the central workstation 10 may further include general-purpose elements other than the elements shown in FIG. 7.

Also, each of the focus pattern setter 110, the parameter calculator 120, the ultrasound generator 130, the region of interest selector 140, the order determiner 150, and the interfaces 160 of the central workstation 10 of FIG. 7 includes and uses one or more processors. Each processor may include an array of logic gates, or a combination of a general-purpose microprocessor and a program that may be executed by the microprocessor. Alternatively, it would be understood by one of ordinary skill in the art that the processor may include any of other types of hardware.

Also, operations of the focus pattern setter 110, the parameter calculator 120, and the ultrasound generator 130 of the central workstation 10 of FIG. 7 are similar to those described above. However, this observed similarity should not be taken as restrictive and these elements of the embodiments may differ as well.

The region of interest selector 140 selects a region of interest to which therapeutic ultrasound is to be radiated. Examples of the region of interest may include a lesion needed to be treated by using ultrasound. For example, the region of interest selector 140 may automatically select the region of interest without the user's intervention, or may select the region of interest by using information about an area that is designated by the user and is input through the interface 160. Regardless of which approach is used, the region of interest selector 140 establishes which volume within object 50 should be targeted with the therapeutic ultrasound.

The region of interest selector 140 transmits information about the selected region of interest to the focus pattern setter 110.

The order determiner 150 determines an order in which therapeutic ultrasounds that form focus patterns are radiated. For example, the order determiner 150 may automatically determine an order in which therapeutic ultrasounds are radiated without the user's intervention, or may determine an order by using information about an order that is designated by the user and is input through the interface 160. For example, the order chosen by the order determiner 150 may be chosen to minimize time consumption or energy usage.

The interface 160 may include a communication interface and a user interface.

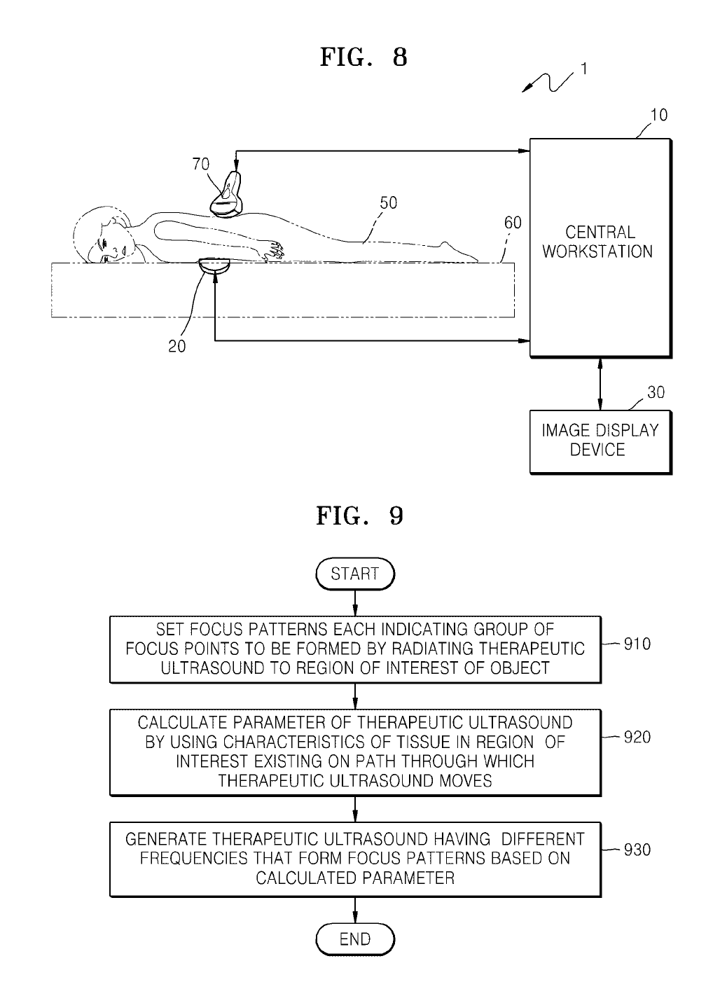

The communication interface transmits information about therapeutic ultrasound to the therapeutic ultrasound probe 20, and receives electrical pulse signals from a diagnostic ultrasound probe 70 which will be explained below. Also, the communication interface transmits an ultrasound image generated by the central workstation 10 to an image display device 30 (see FIG. 8).

The user interface receives information about an order in which a therapeutic ultrasound image is radiated or information about a region of interest to be selected from the user, and transmits the information to the region of interest selector 140 or the order determiner 150. For example, the user interface 160 may include all input/output devices such as a display panel, a mouse, a keyboard, a touch screen, a monitor, and a speaker provided on the central workstation 10. The user interface 160 acts to provide input and output to the user to allow the user to interact with the system and request and obtain results.

FIG. 8 is a diagram illustrating the HIFU system 1 according to another embodiment. The HIFU system 1 includes the central workstation 10 and the therapeutic ultrasound probe 20. In embodiments, the HIFU system 1 further includes the image display device 30 and/or the diagnostic ultrasound probe 70.

Only elements of the HIFU system 1 that are related to the present embodiment are shown in FIG. 8. Accordingly, it would be understood by one of ordinary skill in the art that the HIFU system 1 may further include general-purpose elements other than the elements shown in FIG. 8.

Also, the HIFU system 1 of FIG. 8 is just an example of the central workstation 10 of each of FIGS. 1 and 7. Accordingly, descriptions already made with reference to FIGS. 1 and 7 may apply to the HIFU system 1 of FIG. 8, and thus a repeated explanation will not be given. However, while remarks made with respect to FIGS. 1 and 7 will generally apply to FIG. 8, these remarks should not be taken as limiting and the embodiment of FIG. 8 may also have additional, different, or fewer features than those of FIGS. 1 and 7.

The diagnostic ultrasound probe 70 radiates diagnostic ultrasound to a region of interest, and obtains a reflected ultrasound signal. In detail, the diagnostic ultrasound is partially reflected from layers between various tissues constituting the region of interest. The reflected ultrasound signal oscillates a piezoelectric transducer of the diagnostic ultrasound probe 70, and the piezoelectric transducer outputs electrical pulse signals due to the oscillation. Thus, the diagnostic ultrasound probe 70 uses ultrasound to gather information about the interior of object 50. Rather than focusing ultrasound to kill a tumor, a diagnostic ultrasounds probe 70 uses ultrasound in order to image and scan the subject.

Thus, the diagnostic ultrasound probe 70 may directly generate an ultrasound image of the region of interest by using the electrical pulse signals, or the central workstation 10 may generate an ultrasound image of the region of interest by using the electrical pulse signals. When the diagnostic ultrasound probe 70 directly generates an ultrasound image, the diagnostic ultrasound probe 70 transmits information about the generated ultrasound image to the central workstation 10. When the central workstation 10 generates an ultrasound image, the diagnostic ultrasound probe 70 transmits the electrical pulse signals to the central workstation 10. Thus, the central workstation 10 and the diagnostic ultrasound probe 70 use ultrasound for imagery purposes to gain information for medical treatment.

Also, the diagnostic ultrasound probe 70 and the therapeutic ultrasound probe 20 have a specific positional relationship. In an example, the diagnostic ultrasound probe 70 and the therapeutic ultrasound probe 20 operate by being spaced apart from each other by a predetermined interval or operate by being disposed adjacent to each other. If the therapeutic ultrasound probe 20 and the diagnostic ultrasound probe 70 are located in predetermined positions,

Although the therapeutic ultrasound probe 20 is disposed in the bed 60 in FIGS. 1, 7, and 8, the present invention is not limited thereto. In an example, the therapeutic ultrasound probe 20 is disposed over the object 50 and radiates ultrasound downward. In another example, the ultrasound probe 20 is disposed to one side of the object 50 and radiate ultrasound through the object 50 from one side.

The image display device 30 displays the ultrasound image generated by the central workstation 10. For example, the image display device 30 includes all output devices such as a display panel, a liquid crystal display (LCD) screen, and a monitor provided on the HIFU system 1. In general, any display technology that provides visual feedback may be used as image display device 30. However, central workstation may also use other types of interaction with a user as well, such as audio outputs, printed documents, and the like. Information about the region of interest obtained by the central workstation 10 may be provided to the user through the image display device 30. In some embodiments, the information is used to determine a change in a state, a position, or a shape of tissue.

FIG. 9 is a flowchart illustrating a method performed by the central workstation 10 to generate ultrasound that forms focus points in a region of interest, according to an embodiment.

Referring to FIG. 9, the method includes operations sequentially performed by the central workstation 10 or the HIFU system 1 of FIGS. 1, 7, and 8. Accordingly, although not described, descriptions already made for the central workstation 10 or the HIFU system 1 of FIGS. 1, 7, and 8 apply to and describe the method of FIG. 9.

In operation 910, the focus pattern setter 110 sets focus patterns each indicating a group of focus points to be formed by radiating therapeutic ultrasound to a region of interest of an object. The focus points refer to a plurality of focus points formed when the therapeutic ultrasound probe 20 focuses the therapeutic ultrasound on the region of interest. Focus points are chosen so as to correspond with areas of the object 50 that are designated to receive the maximum intensity of ultrasound radiation so as to cause an increase in temperature. For example, focus points may include parts of tumors that are targeted by the therapeutic ultrasound.

In operation 920, the parameter calculator 120 calculates a parameter of the therapeutic ultrasound by using characteristics of tissue in the region of interest existing on a path through which the therapeutic ultrasound moves. For example, muscle tissue might have a different characteristic from adipose tissue or bone. The path through which the therapeutic ultrasound moves refers to a path through which the therapeutic ultrasound radiated by the therapeutic ultrasound probe 20 moves to the focus points. The focus points may be obtained by using information about the focus patterns transmitted from the focus pattern setter 110. By performing operations 910 and 920, the focus pattern setter 110 and parameter calculator 120 ascertain which foci to target with the ultrasound and how to emit the ultrasound to arrive at the targeted foci.

In operation 930, the ultrasound generator 130 forms the focus patterns and generates therapeutic ultrasounds having different frequencies based on the calculated parameter. The frequencies may be set such that a sound field observed at a point other than the region of interest after the focus patterns are formed is less than a predetermined critical value. For example, the frequencies may be set such that positions or intensities of side lobes or grating lobes generated by the therapeutic ultrasounds that form the focus patterns are not the same. As discussed with respect to FIGS. 6A-6C, if the ultrasound directed towards each focus is emitted with differing frequencies as discussed above, it provides a way to target multiple foci simultaneously, but in a safer manner.

As described above, since the therapeutic ultrasound probe 20 forms focus points in a region of interest of an object, a time taken to treat a lesion distributed over a wide area may be reduced. Also, since the central workstation 10 sets frequencies of therapeutic ultrasounds that form focus patterns to vary according to the focus patterns, the generation of side lobes or grating lobes may be minimized. Also, since therapeutic ultrasounds having different frequencies are radiated, high heat, which may be generated in an area other than the region of interest, may be suppressed, thereby improving safety of HIFU treatment. Therefore, embodiments present a way of using therapeutic ultrasound that improve its safety and efficiency.

Also, a structure of data used in the method may be recorded by using various units on a computer-readable recording medium. Examples of the computer-readable medium include storage media such as magnetic storage media (e.g., read only memories (ROMs), floppy discs, or hard discs), optically readable media (e.g., compact disk-read only memories (CD-ROMs), or digital versatile disks (DVDs)), or PC interfaces (e.g., PCI, PCI-express, or WiFi).

While the present invention has been particularly shown and described with reference to exemplary embodiments thereof by using specific terms, the embodiments and terms have merely been used to explain the present invention and should not be construed as limiting the scope of the present invention as defined by the claims. The exemplary embodiments should be considered in a descriptive sense only and not for purposes of limitation. Therefore, the scope of the invention is defined not by the detailed description of the invention but by the appended claims, and all differences within the scope will be construed as being included in the present invention.

The various units, modules, elements, and methods described above may be implemented using one or more hardware components, one or more software components, or a combination of one or more hardware components and one or more software components.

A hardware component may be, for example, a physical device that physically performs one or more operations, but is not limited thereto. Examples of hardware components include microphones, amplifiers, low-pass filters, high-pass filters, band-pass filters, analog-to-digital converters, digital-to-analog converters, and processing devices.

A processing device may be implemented using one or more general-purpose or special-purpose computers, such as, for example, a processor, a controller and an arithmetic logic unit, a digital signal processor, a microcomputer, a field-programmable array, a programmable logic unit, a microprocessor, or any other device capable of running software or executing instructions. The processing device may run an operating system (OS), and may run one or more software applications that operate under the OS. The processing device may access, store, manipulate, process, and create data when running the software or executing the instructions. For simplicity, the singular term “processing device” may be used in the description, but one of ordinary skill in the art will appreciate that a processing device may include multiple processing elements and multiple types of processing elements. For example, a processing device may include one or more processors, or one or more processors and one or more controllers. In addition, different processing configurations are possible, such as parallel processors or multi-core processors.

As a non-exhaustive illustration only, a terminal/device/unit described herein may be a mobile device, such as a cellular phone, a personal digital assistant (PDA), a digital camera, a portable game console, an MP3 player, a portable/personal multimedia player (PMP), a handheld e-book, a portable laptop PC, a global positioning system (GPS) navigation device, a tablet, a sensor, or a stationary device, such as a desktop PC, a high-definition television (HDTV), a DVD player, a Blue-ray player, a set-top box, a home appliance, or any other device known to one of ordinary skill in the art that is capable of wireless communication and/or network communication.

A computing system or a computer may include a microprocessor that is electrically connected to a bus, a user interface, and a memory controller, and may further include a flash memory device. The flash memory device may store N-bit data via the memory controller. The N-bit data may be data that has been processed and/or is to be processed by the microprocessor, and N may be an integer equal to or greater than 1. If the computing system or computer is a mobile device, a battery may be provided to supply power to operate the computing system or computer. It will be apparent to one of ordinary skill in the art that the computing system or computer may further include an application chipset, a camera image processor, a mobile Dynamic Random Access Memory (DRAM), and any other device known to one of ordinary skill in the art to be included in a computing system or computer. The memory controller and the flash memory device may constitute a solid-state drive or disk (SSD) that uses a non-volatile memory to store data.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A method of generating ultrasound, comprising:
setting focus patterns that each indicate focus points to be formed by radiating therapeutic ultrasound to a region of interest of an object;
calculating a parameter of the therapeutic ultrasound based on characteristics of tissue in the region of interest existing on a path through which the therapeutic ultrasound moves; and
generating therapeutic ultrasound set at different frequencies to form the focus patterns, based on the calculated parameter.

2. The method of claim 1, wherein the different frequencies are set such that the intensity of a sound field at a point other than the region of interest after the focus patterns are formed is less than a predetermined critical value.

3. The method of claim 1, wherein the different frequencies are set such that at least one of positions or intensities of side lobes or grating lobes generated by the therapeutic ultrasound that forms the focus patterns are not the same.

4. The method of claim 1, wherein the calculating of the parameter of the therapeutic ultrasound comprises calculating the parameter of the therapeutic ultrasound based on a density of the tissue, a speed of sound of the therapeutic ultrasound in the tissue, and a wave number of the therapeutic ultrasound in the tissue.

5. The method of claim 1, wherein the parameter of the therapeutic ultrasound is at least one of an amplitude and a phase of the therapeutic ultrasound.

6. The method of claim 1, further comprising selecting the region of interest to which the therapeutic ultrasound is to be radiated,
wherein the setting of the focus patterns comprises setting positions of focus points included in the focus patterns in the selected region of interest.

7. The method of claim 1, further comprising determining an order in which the generated therapeutic ultrasound that forms the focus patterns is radiated.

8. A non-transitory computer-readable recording medium having embodied thereon a program for executing the method of claim 1.

9. An apparatus for generating ultrasound that forms focus points, comprising:
a focus pattern setter configured to set focus patterns each indicating focus points to be formed by radiating therapeutic ultrasound to a region of interest of an object;
a parameter calculator configured to calculate a parameter of the therapeutic ultrasound based on characteristics of tissue in the region of interest existing on a path through which the therapeutic ultrasound moves; and
an ultrasound generator configured to generate therapeutic ultrasound set at different frequencies to form the focus patterns, based on the calculated parameter.

10. The apparatus of claim 9, wherein the ultrasound generator is configured to set the different frequencies such that the intensity of a sound field observed at a point other than the region of interest after the focus patterns are formed is less than a critical value.

11. The apparatus of claim 9, wherein the ultrasound generator is configured to set the different frequencies such that at least one of positions or intensities of side lobes or grating lobes generated by the plurality of therapeutic ultrasounds that forms the focus patterns are not the same.

12. The apparatus of claim 9, wherein the parameter calculator is configured to calculate the parameter of the therapeutic ultrasound based on a density of the tissue, a speed of sound of the therapeutic ultrasound in the tissue, and a wave number of the therapeutic ultrasound in the tissue.

13. The apparatus of claim 9, wherein the parameter of the therapeutic ultrasound is at least one of an amplitude and a phase of the therapeutic ultrasound.

14. The apparatus of claim 9, further comprising a region of interest selector configured to select the region of interest to which the therapeutic ultrasound is to be radiated,
wherein the focus pattern setter is configured to set positions of different focus points included in the focus patterns within the selected region of interest.

15. The apparatus of claim 9, further comprising an order determiner that is configured to determine an order in which the generated therapeutic ultrasound that forms the focus patterns is radiated.
